(19) United States
(12) Patent Application Publication
(10) Pub. No.: US 2025/0011711 A1
(43) Pub. Date: Jan. 9, 2025

(54) PRE-STRESS TREATMENT METHOD FOR REDUCING MORTALITY RATE OF HAEMATOCOCCUS PLUVIALIS

(71) Applicant: NINGBO LUMING BIOTECHNOLOGY CO., LTD., Ningbo City (CN)

(72) Inventors: Luyun CAI, Ningbo City (CN); Ailing CAO, Ningbo City (CN); Ran XU, Ningbo City (CN); Jian LI, Ningbo City (CN)

(21) Appl. No.: 18/634,028

(22) Filed: Apr. 12, 2024

(30) Foreign Application Priority Data
Jul. 7, 2023 (CN) 202310827569.4

(51) Int. Cl. C12N 1/12; A01G 33/00

(52) U.S. Cl. CPC C12N 1/125; A01G 33/00; C12N 2500/60; C12N 2523/00

(57) ABSTRACT

The present invention discloses a pre-stress treatment method for reducing a mortality rate of Haematococcus pluvialis. The method includes the following steps: after the Haematococcus pluvialis is subjected to a logarithmic-growth amplification stage to reach a specific biomass and before the Haematococcus pluvialls enters into an accumulation stage of astaxanthin, performing prestress treatment on the Haematococcus pluvialis, where the prestress treatment indicates adjusting a culture system to include characteristic peaks with wavelength ranges of 430-490 nm and 620-700 nm as spectra parameters. In the present invention, ecological factors in the culture system of the haematococcus pluvialis are adjusted, so that more than 90% of cells therein can detach from flagella in a short time, the walls of the cells are thickened, the cells enter into an ideal immobile cell state in which the astaxanthin can be accumulated, and the mortality rate of the cells is less than 3%, so as to reduce the mortality rate of the cells at an initial stage in which the Haematococcus pluvialis stresses to accumulate the astaxanthin. This reduces the generation of organic matters in an algal liquid, and realizes the purpose of reducing pollution, shortens the accumulation period of the astaxanthin in the Haematococcus pluvialis, and improve the productivity, the quality and yield of the product and the production stability.

### CROSS-REFERENCE TO RELATED APPLICATIONS

The present invention claims priority to Chinese Patent, Patent Application No.: 202310827569.4, filed on 7 Jul. 2023, and the entire contents of which, including but not limited to specification, claims, abstracts, and accompanying drawings are a part of the present invention.

### BACKGROUND OF THE INVENTION

### Field of the Invention

The present invention relates to the field of algal biotechnology, and in particular, to a pre-stress treatment method for reducing a mortality rate of Haematococcus pluvialis.

### Description of the Related Art

Culture of Haematococcus pluvialis in large scale is currently recognized as a best source to produce natural astaxanthin. Generally, high astaxanthin is obtained through artificial culture of Haematococcus pluvialis in two stages: firstly, a large amount of Haematococcus pluvialis cells are subjected to expanding culture to obtain sufficient biomass, which needs to provide the preferred conditions suitable for the vegetative growth and division and proliferation of the cells; secondly, unfavorable internal and external conditions are created to induce the cells to accommodate astaxanthin under stress, entering into an astaxanthin enrichment stage. Commonly imposed unfavorable conditions include nutrient deprivation, high light, high temperature, and the like. For example, patent: a method for culturing a haematococcus organism for producing astaxanthin (Publication No. CN108350478A) discloses a culture method of adherent Haematococcus pluvialis, and the method includes two steps: a first step is initial culture, that is, cells arrayed on the surface of the substrate are exposed to a low-luminous energy for culture; a second step is that the cells are exposed to a more luminous energy relative to the previous step to induce the synthesis of astaxanthin. Furthermore, a method for inducing Haematococcus pluvialis to produce astaxanthin by adding a chemical reagent is provided, for example, patent: a culture method of Haematococcus pluvialis (Publication No.: CN109609385A); in the culture method, according to the culture characteristics of the Haematococcus pluvialis in a growth stage and an induction stage and based on a basic culture solution, NaHCO₃, ampicillin and vitamin B12 are added to the culture solution in the growth stage; and NaAc, gibberellin and the like are added to the culture solution in the induction stage, to improve the yield of the Haematococcus pluvialis and the content of astaxanthin. In addition, phytohormones such as jasmonic acid, brassinolide, fulvic acid, and naphthylacetic acid are utilized to induce astaxanthin to accumulate in the Haematococcus pluvialis, such as patents CN101974600A, CN101974599A, CN109679853A, and CN107338194A.

The Haematococcus pluvialis is very sensitive to environmental change, has the poor ability to resist bacterial and protozoan pollution in the vegetative growth period, and loses its reproductive ability in extreme environment, resulting in difficulty in establishing a stable and efficient production technology system.

Culture of Haematococcus pluvialis to produce astaxanthin has considerable technical difficulties in algae species, design of photobioreactors, high-density culture conditions of cells, ecological control technologies of staxanth in accumulation and the like. The process in which astaxanthin accumulates in the Haematococcus pluvialis cells is the adaptive protection of algae cells to unfavorable environment conditions. Therefore, when people apply induced pressure according to this principle, the use of any of the above methods will inhibit the normal growth of cells, disrupt their original life rhythm and metabolic process and cause a large number of dead cells. The dead cells will not only affect the final yield, but also cause the rapid reproduction of polluted organisms such as microorganisms and protozoa with the release of organic matters from cell lysis, which will affect the culture process and seriously lead to the failure of culture.

Therefore, how to quickly and smoothly redden cells, reduce damage to the cells arising from stress factors as much as possible, and reduce the mortality rate of the cells during stress induction is a considerable problem during cultivation of the Haematococcus pluvialis. For the problem, a clear concept and an operation method have not yet been proposed in the prior art.

### BRIEF SUMMARY OF THE INVENTION

In order to overcome the defects of the prior art, an intermediate prestress treatment process is added at the final segmented culture stage of algae cells of Haematococcus pluvialis and before the algae cells enter into an astaxanthin accumulation stage, and the prestress method indicates adjusting external conditions of a culture system between the proliferation stage and the astaxanthin accumulation stage during cultivation of the Haematococcus pluvialis. Culture conditions are adjusted, so that the algae cells enter into the astaxanthin accumulation stage in a more excellent state to shorten the production cycle of astaxanthin accumulation and synchronously develop the cells in the culture system; this reduces the mortality rate of the cells caused by the artificial stress conditions and avoids pollution, helping improve the quality, yield, and production stability of the product in large-scale production.

In the prior art, fluorescent lamps or sunlights are used as a light source for stress treatment. As the light source has heterogeneous spectrum, luminous energy that is within a wavelength range and strongly lethal to the algal cells is high, while luminous energy that is suitable for use in stress and within the wavelength range is insufficient, which often leads to the high mortality rate of the algal cells in a stress process; for this, the present invention introduces a prestress stage and systematically studies the adaptability of the Haematococcus pluvialis to various wavelengths of the spectrum, so that two characteristic peaks at 430-490 nm and 620-700 nm are identified and can exert the optimal effect in prestress treatment; after the cells are subjected to the prestress treatment, the present invention can efficiently reduce the mortality rate without affecting the productivity.

The present invention achieves the above purpose by the following technical solutions:

A pre-stress treatment method for reducing a mortality rate of Haematococcus pluvialis is provided, and the method includes: prestressing the Haematococcus pluvialis subjected to a logarithmic-growth proliferation stage, where external conditions for the pre-stress treatment method include characteristic peaks with wavelength ranges of 430-490 nm and 620-700 nm as spectra parameters.

Further, a cell density of the Haematococcus pluvialis subjected to the logarithmic-growth proliferation stage is 3-4×10⁵ cells/mL. The logarithmic growth phase is a common concept in the prior art, namely, a rapid growth and reproduction stage of algal cells; for this application, after the Haematococcus pluvialis rapidly grows, and an algae cell density is more than 30×10⁴ cells/mL, that is, the cell density of the Haematococcus pluvialis subjected to the logarithmic-growth proliferation stage is higher than 30×10⁴ cells/mL, the astaxanthin accumulated in the Haematococcus pluvialis of the present invention is subjected to prestress treatment.

In some embodiments, the cells subjected to the logarithmic growth phase are generally not required for the stress treatment. However, in order to obtain a maximum biomass, the cells are usually allowed to grow close to a plateau stage before the stress treatment. The so-called plateau stage means that, in a specific volume, when the concentration of inoculated cells is very low, the cells are cultured for a specific period and in the plateau stage after subjected to the logarithmic growth phase; in this case, the number of cells substantially remain unchanged, and the cells are not subjected to logarithmic division, and the cells in the volume reach a maximum concentration. In this case, the stress treatment is performed, which can increase the yield of astaxanthin per unit volume. It can be understood that cells at any time can be subjected to the stress treatment. Therefore, in a case that the cell density or concentration in the above embodiments is 30×10⁴ cells/mL, the cells are not substantially subjected to logarithmic division, but are in a relatively stable state; and this concentration is similar to a maximum concentration.

Further, the characteristic peaks with the wavelength ranges of 430-490 nm and 620-700 nm have a light intensity ratio of 0.1-1:1.

In some embodiments, the characteristic peaks with the wavelength ranges of 430-490 nm and 620-700 nm have a light intensity ratio of 0.7:1.

Further, the external conditions for the pre-stress treatment method further include: an algal liquid with the cell density of 1×10⁴ cells/mL having alight intensity of 300-400 Lux.

Further, the external conditions for the pre-stress treatment method further include: a photoperoid L:D of 16-18:6-8.

In some embodiments, a photoperoid L:D is 16:8.

Further, a photobioreactor is used as a light source for the pre-stress treatment method.

Further, the external conditions for the pre-stress treatment method further include: a temperature of 26° C.-28° C. and a pH of 8.0-8.5.

In some embodiments, the temperature is 28° C. and the pH is 8.2.

Further, a treatment time for the pre-stress treatment method is 24-36 hours.

In some embodiments, a treatment time for the pre-stress treatment method is 32 hours.

More than 90% of the algal cells subjected to prestress treatment can reach the state of entering the astaxanthin accumulation stage in a short time, and the mortality rate of the cells is less than 3%; the state of entering the astaxanthin accumulation stage after the prestress treatment means that more than 90% of the algal cells are capable to exhibit at least two of the following characteristics: a. original motile cells losing flagella and becoming immobile cells; b. thickening of walls of the cells; c. reddening in the centers of the cells and accumulation of a small amount of astaxanthin therein.

Preferably, more than 90% of the algal cells are also capable to exhibit the following characteristics: d. the cells changing from an original water drop shape to a spherical shape; e. the increase of the cells in size and diameter (from 20-25 μm to 30-40 μm).

The logarithmic growth phase is a common concept in the prior art, namely, a rapid growth and reproduction stage of algal cells; for this application, after the haematococcus pluvialis rapidly grows, and an algae cell density is more than 30×10⁴ cells/mL, that is, the cell density of the Haematococcus pluvialis subjected to the logarithmic-growth proliferation stage is higher than 30×10⁴ cells/mL, the astaxanthin accumulated in the Haematococcus pluvialis of the present invention is subjected to prestress treatment.

The present invention has the following beneficial effects:

1. The present invention provides a pretreatment method used between the logarithmic-growth proliferation stage and the astaxanthin accumulation stage, namely, a prestress stage; prestress treatment indicates that the algae cells are subjected to stress adaptation induction by adjusting the external conditions of the culture system, thereby solving the problem of damage to cells in an existing culture method that the cells directly enter into the formal reddening promotion stage; the prestress treatment can reduce the mortality rate of the cells in an initial induction stage and ensure the productivity and process stability, avoiding contamination from exogenous sources such as protozoa and microorganisms due to lysis of dead cells, which greatly reduces the risk of production failure and improves the quality of products.

2. In the present invention, the external conditions adapted to the prestress of the algae cells are screened out through many tests, so that the mortality rate of the algae cells at an initial formal stress stage is reduced. This is because when the cells not prestressed in the prior art directly enter the formal reddening promotion stage, the culture environment of algae changes rapidly due to the rapid entry into the reddening promotion stress environment, individual cells exhibit quite different performance, some cells can quickly form thick-walled cells and begin to accumulate astaxanthin, and some cells will die from inhibition or even lysis; the drastic change in conditions is not conducive to the consistency of the overall level of the cells and the subsequent stable yield of the cells, especially for a biomass indicator. However, the cells subjected to prestress treatment in the present invention usually have a same cell size and low mortality rate, thus achieving the high collection effect of biomass. In the prior art, when the cells directly enter into the stress stage, the mortality rate of the early cells is at least 30% according to the different induction pressures and modes, while the mortality rate of the algae cells prestressed in the present invention can be substantially reduced below 3% with the change in cultivation conditions.

3. The pretreatment method provided by the present invention is particularly important for those algae strains that are susceptible to the change in external environmental conditions and can quickly accumulate a high content of astaxanthin, but have poor resistance and are prone to cause damage or death due to the change in conditions, so that the algae strains with a high content of astaxanthin accumulated quickly can be produced and applied; meanwhile, the present invention also plays a better role in expanding the induction mode used in the induction process of the astaxanthin in the Haematococcus pluvialis, and can alleviate the phenomenon of cell death caused by the induction pressure to the greatest extent when a strong and effective induction mode is adopted.

### BRIEF DESCRIPTION OF THE DRAWINGS

FIG. 1 shows a microscopic image of Haematococcus pluvialis cells before prestress treatment; and

FIG. 2 shows a microscopic image of Haematococcus pluvialis cells after prestress treatment.

### DETAILED DESCRIPTION OF THE INVENTION

A pretreatment method for astaxanthin accumulated in Haematococcus pluvialis according to the present invention is further explained with reference to specific embodiments and accompanying drawings, but the contents of the following embodiments should not be construed as a limitation of the scope of protection of the present invention. Unless otherwise specified, reagents used in the embodiments of the present invention are commercially available.

The original species of the Haematococcus pluvialis used in the following embodiments were purchased from Wuhan Institute of Hydrobiology, Chinese Academy of Sciences, and their data were tested in the following manner:

The following mortality rates were the ones after the Haematococcus pluvialis was stressed in an early production stage of astaxanthin; the mortality rate of cells was tested by using an algae counting chamber and a counter to count the percentage of dead cells in the total cells, where the counting chamber had a total capacity of 100 μL and was divided into 100 counting cells; after sampling, algal cells were evenly distributed, randomly selected and counted under a microscope in 25-50 cells, where statistical indicators in each cell included total number of cells, normal cells, dead cells, and successfully prestressed cells (with at least two or more of the characteristics: loss of flagella, thickening of walls of the cells, and reddening of the centers of the cells). The dead cells indicate cells that are lysed or have dispersive interiors. The mortality rate of cells was calculated based on: the number of dead cells÷the total number of cells×100%.

The immobility rate of cells and the mortality rate of cells had a similar calculation way; the former was calculated based on: the number of successfully prestressed cells (with at least two or more of the characteristics: loss of flagella, thickening of walls of the cells, and reddening of the centers of the cells)÷the total number of cells×100%.

The diameter of the cell was determined by using the algae counting chamber, the counter, and a micrometer, where the counting chamber had a total capacity of 100 μL and was divided into 100 counting cells; after sampling, algal cells were evenly distributed, randomly selected and counted under a microscope in 25-50 cells; then, the diameter of the cell was determined by using the micrometer, counted and averaged to obtain the average diameter of the cell.

The astaxanthin content was determined by using liquid chromatography; the cells were crushed by using a test tube disperser; pigment components therein were fully extracted with methyl alcohol: dichloromethane=3:1 solution as an extract; and the extract was saponified by a methanol solution containing 0.1 mol/L potassium hydroxide for 4 h and then determined, where the chromatographic conditions were set as follows: column temperature 25° C., injection volume 10 μL, flow rate 1 ml/min, and wavelength 476 nm; a mobile stage was a mixed solution of methanol: dichloromethane: acetonitrile: water=85:5:5:5; and a chromatographic column was C18. Liquid chromatography standards were purchased from Sigma Company.

The dry weight of the cells was tested through the steps: drying the cells in an oven at 105° C. for 2.5 h to a constant weight, transferring the dried cells into a desiccator, cooling them to ambient temperature, and weighing them with an electronic analytical balance to obtain data; the dry weight was calculated based on a formula: W=(m2−m1)/v, where w denotes dry weight data, m1 denotes container weight, m2 denotes the total weight of the container and the algal liquid after drying, and v denotes the volume of the sampled algal liquid.

### Embodiment 1: Culture Method for Astaxanthin from Haematococcus pluvialis Subjected to Prestress Treatment

### 1. Introduction to Algal Species in an Algal Liquid

### 1.1 Device Preparation

In this embodiment, a plate-type built-in light source photobioreactor and supporting detection and control devices thereof were used as a culture system of Haematococcus pluvialis; the culture system, including pipes, valves and other devices contacting with the culture were sterilized; various monitoring probes and auxiliary devices were calibrated; 500 L and 1000 L tanks were selected, where 500 L tanks were used in a logarithmic-growth proliferation stage and an early reddening promotion stage, 1000 L tanks were used in an astaxanthin accumulation stage, and the tanks were connected through the pipes and the valves. The plate-type built-in light source photobioreactor is a common device for culturing cells. These devices are commercially available for purchase, and the purposes of the present invention can be realized through any commercially available devices. The devices of the present invention were purchased from Shanghai Guangyin Biotechnology Co. Ltd, with product model GY-FYQ-DT-1000 L and GY-FYQ-DT-500 L. The light source was an LED light source.

After the culture system of the 500 L tank was prepared, purified water was added to its water level, and a gas flow meter was turned on to pump gas (gas mixture of air and carbon dioxide). The parameters of a 500 L bioreactor were set as follows: light intensity 6,000 Lux, photoperoid L:D 12:12, temperature controlled at 21° C.-26° C., pH controlled at 7.0-7.5, and gas mixture of compressed air and carbon dioxide as introduced gas, where the carbon dioxide accounts for 3.5% of the introduced gas, and the gas flow rate is 4 m³/h.

### 1.2 Preparation of Culture Solution

An optimized culture formula of Haematococcus pluvialis was used in a culture medium; during production, a mother solution (a high-concentration nutrient salt solution, which is added into a reactor in proportion according to the required concentration in the reactor) was sequentially added in the 500 L bioreactor in proportion, and continuously aerated and stirred. The concentration of each nutrient in a final culture solution was 0.15 g/L sodium nitrate, 7.5×10⁻³ g/L dipotassium hydrogen phosphate, 1.75×10⁻² g/L potassium dihydrogen phosphate, 3.7×10⁻² g/L magnesium sulfate, 1.9×10⁻² g/L calcium chloride, 1.14×10⁻² g/L boric acid, 8.82×10⁻³ g/L zinc sulfate, 1.44×10⁻³ g/L manganese chloride, 1.2×10⁻³ g/L sodium molybdate, 1.57×10⁻³ g/L copper sulfate, 4.9×10⁻⁴ g/L cobalt nitrate, 5.0×10⁻² g/L ethylene diamine tetraacetic acid, and 4.98×10⁻³ g/L ferrous sulfate.

### 1.3 Introduction to Algae Species

Algae species were screened, and the screened algae species were characterized by rapid proliferation, sensitivity to external stimulation, rapid initiation of astaxanthin accumulation, and high dry weight of cell and astaxanthin content. In practical operation, the algae species were screened through microscopic examination; the cells screened through microscopic examination exhibited active swimming and obvious phobophototaxis, with full intracellular chromatophores and moderate size; the algae cells with an average diameter of 25 μm were used as the algae species; the culture system was free from contaminants and used for culturing the algae species; then, the algae species with an initial density of 2×10⁴ cells/mL were introduced into the mother solution; after inoculation, the cell density in the tank was determined as being 2.41×10⁴ cells/mL.

It can be understood that any other suitable algae species is feasible; in actual industrialization, the selection of excellent algae species facilitates the quality and yield of astaxanthin in a later period, but this does not mean that other algae species cannot be used in the present invention.

### 2. Logarithmic-Growth Proliferation Stage

The introduced algae species were subjected to proliferation culture in the logarithmic stage, and the parameters of the 500 L bioreactor in 1.1 were considered as culture conditions, including light intensity 6000 Lux, photoperoid L:D 12:12, temperature controlled at 21° C.-26° C., pH controlled at 7.0-7.5, and gas mixture of compressed air and carbon dioxide as introduced gas, where the carbon dioxide accounts for 3.5% of the introduced gas, and the gas flow rate is 4 m³/h. A carbon dioxide gas pipe valve was automatically started and closed according to acidity and alkalinity values of the culture system detected by a pH probe; when pH in the culture system was higher than a preset value (7.0-7.5), the introduction to the carbon dioxide was stopped, the compressed air and the carbon dioxide were subjected to front-end sterile filtration and drying treatment, and the samples were daily taken for microscopic examination to observe a cell growth situation; until the fourth day, the microscopic examination revealed division of multiple cells and generation of multiple new cells, with active cell swimming, full chromatophores, and clean and unpolluted culture system; meanwhile, the cell counting results on the fourth day was 35.2×10⁴ cells/mL, indicating that the cells can enter into the early reddening promotion stage at the moment.

### 3. Pre-Stress Treatment Method (Early Reddening Promotion Stage)

The cells entered into the early reddening promotion reddening stage after subjected to the logarithmic-growth proliferation stage. The cells were subjected to the early reddening promotion stage still in the original culture system used in the logarithmic-growth proliferation stage, that is, the cells were subjected to the early reddening promotion stage in the 500 L bioreactor. The bioreactor was adjusted to the following culture conditions: the characteristic peaks with the wavelength ranges of 430-490 nm and 620-700 nm as the spectra parameters being used as the light source, the characteristic peaks having the light intensity ratio of 0.7:1, a total light intensity of 12,000 Lux, a photoperoid L: D of 16:8, a temperature controlled at 26° C.-28° C., and a pH controlled at 8.0-8.2; in this specific embodiment, the temperature was 28° C. and the pH was 8.2. After the algae cells were cultured for 36 hours under this condition, most of them were sampled for microscopic examination. During the microscopic examination, when more than 80% of algae cells exhibited at least two of the following three characteristics, the cells completed the early reddening promotion stage and became standard cells. The so-called three characteristics are: a. original motile cells losing flagella and becoming immobile cells; b. thickening of walls of the cells; c. accumulation of astaxanthin in the centers of the cells. Characteristics a, b and c are the main indicators to judge whether early forced reddening is completed. Of course, there are also two auxiliary indicators, namely: d. the cells changing from an original water drop shape to a spherical shape; e. the increase of the cells in size and diameter (from 20-25 μm to 30-40 μm).

When standard cells were observed after 36 hours, the early reddening promotion was completed; if the standard cells were not observed, the cells would continue to be cultured and tested once every 12 hours until the standard cells were observed. The standard cells herein mainly indicate that the cells are the standard cells when meeting two of the characteristics a, b and c; of course, the cells meeting the three characteristics is also a preferred specific embodiment; in some embodiments, the cells meeting the three characteristics and two auxiliary indicators is also a preferred embodiment.

In the specific embodiment, after the cells were cultured for 36 hours, the standard cells were examined under a microscope, exhibiting the following characteristics: no division of the cells, the small number of new cells, great reduction in the proportion of motile cells, and reduction in the proportion of green cells in the total cells in the logarithmic-growth proliferation stage from 87% to 18%, more than 80% of algae cells losing their flagella and becoming immobile cells, the increased volume of the cells, and increase in the diameters of the cells from 25 μm to 30 μm on average (some of the cells exceed 40 μm in diameter and account for about 7% of the total cells), change in the cell shape from an original water drop shape to a spherical shape, the walls of the cells being clearly visible under the microscope and obvious thickened, and reddening at the centers of some cells. The above characteristics show that the algae cells can terminate the early reddening promotion stage and enter the next astaxanthin accumulation stage.

The cells are subjected to the above-mentioned early reddening promotion stage in the original culture system in the logarithmic-growth proliferation stage; the device is not additionally provided, and the solution of algae is not transferred and sedimented; under the normal culture condition, ecological factors are adjusted to realize the early reddening promotion stage, which facilitates large-scale production and application of cells; in addition, when the cells enter into the early reddening promotion stage, nutrient elements such as nitrogen and phosphorus in the culture system are substantially consumed, facilitating rapid reddening induction of the cells with the stress factor of deficient nutrient salts, which shortens the reddening induction period, increases two important collection indicators such as the dry weight of the cell and the astaxanthin content, saves equipment and labor costs, and reduces energy consumption and the uncertain risk factors caused by many operations.

### 4. Astaxanthin Accumulation Stage (Formal Reddening Promotion Stage) and Collection of Solution of Algae

After the early reddening promotion stage, according to the two-stage production process, the solution of algae was transferred from the 500 L bioreactor to the 1000 L bioreactor for astaxanthin accumulation, and the production cycle of astaxanthin in the accumulation stage was generally within 10 days; the 1000 L bioreactor had the following culture conditions: monochromatic lights emitted by an LED light source including three characteristic peaks with wavelengths of 370-420 nm, 400-495 nm and 615-700 nm respectively, where the three characteristic peaks has the light intensity ratio of 1:50:70, the light intensity of 35,000 Lux, and the photoperoid L: D of 24:0, with the temperature controlled at 28° C.-30° C., the pH controlled at 8.5-9.0, and the gas flow rate of 4 m³/h; here, the temperature was controlled at 28° C. and the pH was controlled at 8.8. The samples were daily taken for microscopic examination; the microscopic examination results showed that the culture system was clean and unpolluted, the cells gradually increased in volume, the diameters of most cells exceeded 40 μm, and the colors of the cells continued to deepen from their centers to the entire cells until the cells presented dark red; the protochlorophyll color was substantially invisible, the solution of algae looked dark red, and the cells sunk after standing, indicating that the astaxanthin has been accumulated and can be collected.

In the embodiment, the microscopic examination on the seventh day showed that the solution of algae met the standard, and the solution of algae was collected, sedimented and centrifuged to obtain an algae mud, and the algae mud was freeze-dried to obtain a Haematococcus pluvialis powder enriched in astaxanthin. The mortality rate of the cells determined at the 24th hour at the initial astaxanthin accumulation stage was 0.6%; during collection, the astaxanthin content and dry weight of the algae cells were determined. The results showed that the astaxanthin content was 4.89% and the dry weight was 1.03 g/L. The Haematococcus pluvialis was subjected to buffering treatment when entering into the astaxanthin accumulation stage, so that more than 80% of the cells therein could enter an ideal reddening promotion state in a short time, to reduce the mortality rate of the cells and avoid pollution in the initial reddening promotion stage.

### Embodiment 2: Comparison of Cells Subjected to Prestress Treatment or not

In the embodiment, two groups of tests were set up, the method described in Embodiment 1 was used in one group, while the pre-stress treatment method was removed in the other group based on Embodiment 1 (the third step in Embodiment 1 was omitted); the cells in each group were tested in triplicate to determine their mortality rate at the 24th hour at the initial astaxanthin accumulation stage; during collection, the astaxanthin content and dry weight of the algae cells were determined, with the results shown in Table 1.

TABLE 1
Comparison of cells subjected to prestress treatment or not

| | | Mortality | | |
|---|---|---|---|---|
| | | rate of | Astaxanthin | Dry weight |
| Classification | No. | cells (%) | content (%) | (g/L) |
| Non-prestress | 1 | 15.5 | 1.24 | 0.44 |
| group | 2 | 15.0 | 1.25 | 0.47 |
| | 3 | 14.8 | 1.32 | 0.53 |
| | Average value | 15.1 | 1.27 | 0.48 |
| Prestress group | 1 | 0.6 | 4.81 | 1.01 |
| | 2 | 0.6 | 4.94 | 1.04 |
| | 3 | 0.6 | 4.92 | 1.03 |
| | Average value | 0.6 | 4.89 | 1.03 |

It can be seen from the test results that: in the non-prestress group, the mortality rate of the cells is relatively high, and the astaxanthin content and the dry weight are low; in the group subjected to prestress treatment, the mortality rate of the cells is only 0.6%, and the astaxanthin content and the dry weight are high, which is beneficial to production.

In addition, the algae cells not subjected to prestress treatment may be intolerant to the environment when entering into the astaxanthin accumulation stage, leading to the death of large-scale cells (mortality rate of cells >30%), which will lead to the failure of astaxanthin production. In order to quantitatively evaluate how much failure probability can be reduced by prestress, we set up 50 groups in each of the above two groups of tests. The results were as follows: 11 groups in the non-prestress group failed to complete production, with a failure rate of 22%; the prestress group all completed the production. Obviously, the prestress treatment can also reduce the failure rate of astaxanthin production.

### Embodiment 3: Screening of Wavelength Ranges

In the prior art, sunlights or LED white lights are often used as a light source for stress and mixed lights with mixed wavelengths, and the stress results in the high mortality rate of algae cells or the poor accumulation effect of astaxanthin in the algae cells. Under normal sunlights, the mortality rate is often as high as 15%-30%, even more than 30%, and the high mortality rate seriously affects the yield of astaxanthin. Therefore, the wavelengths may be an important factor to affect the survival of the algae cells.

In the embodiment, we selected a plate photobioreactor with an external airlift light source and its supporting detection and control devices as a Haematococcus pluvialis culture system, calibrated all monitoring probes and auxiliary devices, and selected 10 groups of photobioreactors with a 100 L culture volume to carry out culture experiments. The solution of algae used is clean green swimming Haematococcus pluvialis in the logarithmic growth phase, and the density thereof is 30×10⁴ cells/mL. Methods used during the algae introduction and the logarithmic-growth proliferation stage are the same as those in Embodiment 1.

The photobioreactor were adjusted to the following external conditions: light intensity 12,000 Lux, photoperiod L: D 16:8, temperature controlled at 28° C. (automatically controlled by a temperature controller), and pH controlled at 8.2 (controlled by adjusting the content of carbon dioxide); according to previous studies, the absorption spectrum of the algae cells is in the range of 400-700 nm, so we set the spectra parameters as the wavelength ranges of 400-430 nm, 430-460 nm, 460-490 nm, 490-520 nm, 520-550 nm, 550-580 nm, 580-610 nm, 610-640 nm, 640-670 nm, and 670-700 nm respectively. Under the above conditions, the cells were subjected to the prestress treatment for 36 h, entered into the astaxanthin accumulation stage (formal reddening promotion stage) and the solution of algae collection stage in Embodiment 1; the mortality rate of the cells was determined at the 24th hour at the initial astaxanthin accumulation stage; during collection, the astaxanthin content and dry weight of the algae cells were determined, with the results shown in Table 2.

TABLE 2
Wavelength and mortality rate of cells

| Wavelength | Mortality rate | Astaxanthin | Dry weight |
|---|---|---|---|
| range (nm) | of cells (%) | content (%) | (g/L) |
| 400-430 | 9.6 | 1.30 | 0.54 |
| 430-460 | 2.7 | 2.73 | 0.80 |
| 460-490 | 1.9 | 3.28 | 0.83 |
| 490-520 | 10.7 | 1.35 | 0.55 |
| 520-550 | 11.9 | 1.31 | 0.56 |
| 550-580 | 12.4 | 1.33 | 0.55 |
| 580-610 | 8.4 | 1.35 | 0.57 |
| 610-640 | 3.5 | 2.37 | 0.72 |
| 620-640 | 2.8 | 2.77 | 0.80 |
| 640-670 | 2.4 | 2.89 | 0.82 |
| 670-700 | 1.6 | 3.43 | 0.86 |

The results show that the mortality rate of the algae cells is generally less than 3% in the wavelength range of 430-490 nm and 620-700 nm, and the algae cells exhibit the following characteristics: no division of the cells, the small number of new cells, great reduction in the proportion of motile cells, and reduction in the proportion of green cells in the total cells in the logarithmic-growth proliferation stage from 87% to 18%, more than 95% of algae cells losing their flagella and becoming immobile cells (with the mortality rate of about 2%), the increased volume of the cells, and increase in the diameters of the cells from 25 μm to 35 μm on average (some of the cells exceed 40 μm in diameter and account for about 7% of the total cells), change in the cell shape from an original water drop shape to a spherical shape, the walls of the cells being clearly visible under the microscope and obvious thickened, and reddening at the centers of some cells. The above characteristics show that the algae cells complete the prestress stage and enter into the next astaxanthin accumulation stage.

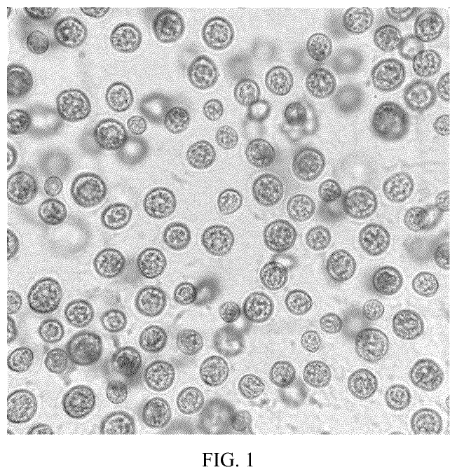

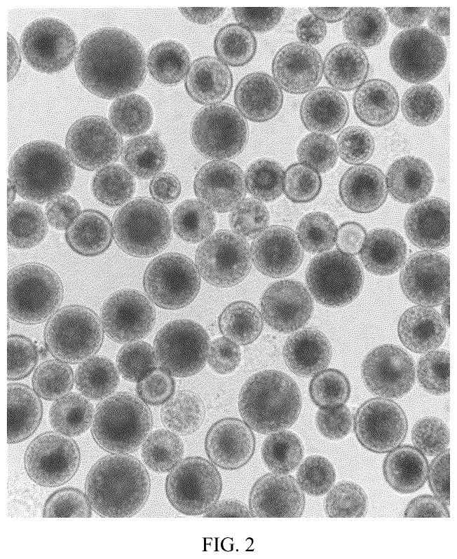

Microscopic images of some Haematococcus pluvialis cells before and after subjected to prestress treatment for 36 hours are shown in FIG. 1 and FIG. 2. FIG. 1 shows the characteristics of the Haematococcus pluvialis before subjected to pretreatment, and FIG. 2 shows the following characteristics of the cells after subjected to prestress treatment: a. original motile cells losing flagella and becoming immobile cells; b. thickening of walls of the cells; c. reddening at the centers of the cells.

In addition, through the determination of the astaxanthin content and dry weight of the algae cells during collection, we can also find the high astaxanthin content and high dry weight in the wavelength ranges of 430-490 nm and 620-700 nm, that is, the astaxanthin yield is significantly increased; however, the accumulation and production effect of the astaxanthin is not obviously increased when lights with other wavelengths are subjected to prestress treatment, but the astaxanthin content is not high and the dry weight is low, and the astaxanthin yield is close to that obtained by a production scheme in which the cells are not subjected to prestress treatment.

The cells are subjected to the above-mentioned prestress treatment stage in the original culture system in the logarithmic-growth proliferation stage; the device is not additionally provided, and the solution of algae is not transferred and sedimented; under the normal culture condition, ecological factors are adjusted to realize the prestress treatment stage, which facilitates large-scale production and application of cells; in addition, when the cells enter into the prestress treatment stage, nutrient elements such as nitrogen and phosphorus in the culture system are substantially consumed, facilitating rapid accumulation induction of the cells with the stress factor of deficient nutrient salts, which shortens the induction period, increases two important collection indicators such as the dry weight of the cell and the astaxanthin content, saves equipment and labor costs, and reduces energy consumption and the uncertain risk factors caused by many operations.

### Embodiment 4: Light Mixing Ratio at Wavelengths of 430-490 nm and 620-700 nm

In Embodiment 3, we screen two characteristic peaks that can reduce the mortality rate of the cells to below 3% and are within the wavelength ranges of 430-490 nm and 620-700 nm. Because their mechanisms of enhancing the prestress effect are different, the combination of such characteristic peaks that can produce good effects on prestress can achieve good results. Therefore, in this embodiment, we study the light intensity ratio of such characteristic peaks by using a same test method as in Embodiment 3, the only difference is that two characteristic peaks with wavelength ranges of 430-490 nm and 620-700 nm are used as a fixed light source to change the light mixing ratio thereof. Similarly, after subjected to the prestress treatment for 36 hours, the cells enter into the astaxanthin accumulation stage (formal reddening promotion stage) and the solution of algae collection stage described in Embodiment 1. The mortality rate of the cell is determined at the 24th hour at the initial astaxanthin accumulation stage; during collection, the astaxanthin content and dry weight of the algae cells are determined, with the results shown in Table 3.

TABLE 3
Light mixing ratio at wavelengths of 430-490 nm and 640-700 nm

| Light intensity | Mortality rate | Astaxanthin | Dry weight |
|---|---|---|---|
| ratio | of cells (%) | content (%) | (g/L) |
| Only 620-700 nm | 2.5 | 2.89 | 0.80 |
| 0.1 | 1.4 | 3.56 | 0.89 |
| 0.5 | 1.1 | 4.12 | 0.96 |
| 0.7 | 0.6 | 4.89 | 1.03 |
| 0.9 | 1.2 | 4.07 | 0.94 |
| 1.0 | 1.8 | 3.46 | 0.86 |
| 1.1 | 2.2 | 3.16 | 0.84 |
| 1.5 | 2.3 | 2.97 | 0.84 |
| Only 430-490 nm | 2.6 | 2.81 | 0.80 |

The results show that when the characteristic peaks with the wavelength ranges of 430-490 nm and 620-700 nm have the light intensity ratio of 0.1-1:1, the mortality rate of the algae cells may be lower than 2%; when the characteristic peaks with the wavelength ranges of 430-490 nm and 620-700 nm have the preferred light intensity ratio of 0.7:1, the mortality rate of the algae cells is only 0.6%, with the highest astaxanthin content and dry weight, and the best production effect of astaxanthin.

It can be seen that after mixed in a certain proportion, the characteristic peaks with the wavelength ranges of 430-490 nm and 620-700 nm have the higher effect of reducing the mortality rate of the algae cells than a single characteristic peak, with a synergistic effect; in addition, the single characteristic peak cannot achieve the illumination effect caused by a combination of the characteristic peaks, and only the combination of such characteristic peaks can achieve the above effect.

### Embodiment 5: Screening of Light Intensity

In actual production, the light intensity has a significant impact on the mortality rate, immobility rate and average diameter of the Haematococcus pluvialis cells. If the light intensity is too high, the mortality rate will increase; although the increase in the immobility rate and the average diameter of the cells promotes the photosynthesis of cells, the high mortality rate is obviously not conducive to the production of the cells. The low light intensity leads to the poor stress effect and the low mortality rate, but the immobility rate of the cell is also low, and the average diameter will not increase significantly, which is also not conducive to the production of the cells. Based on the above problems, the suitable light intensity for the pre-stress treatment method is studied and selected in the embodiment.

In the embodiment, a plate photobioreactor with an external airlift light source and its supporting detection and control devices were used as a Haematococcus pluvialis culture system, all monitoring probes and auxiliary devices were calibrated, and several groups of photobioreactors with a 100 L culture volume were selected to carry out culture experiments. The solution of algae used is clean green swimming Haematococcus pluvialis in the logarithmic growth phase; and the density thereof is 30×10⁴ cells/mL. Methods of algae introduction and proliferation in the logarithmic growth phase are the same as those in Embodiment 1.

The photobioreactor were adjusted to the following external conditions: the characteristic peaks with the wavelength ranges of 430-490 nm and 620-700 nm as the spectra parameters, the characteristic peaks having the light intensity ratio of 0.7:1, and the photoperiod L:D of 16:8, temperature controlled at 28° C. and pH controlled at 8.2; the light intensity was adjusted so that the solution of algae with the cell density of 1×10⁴ cells/mL had the following illumination intensities: 250 Lux, 300 Lux, 350 Lux, 400 Lux, and 450 Lux; the total light intensity=algal cell density*light intensity per unit density; the algal cells were subjected to prestress treatment for 36 hours and then entered into the astaxanthin accumulation stage in the same method as in Embodiment 1. At the 24th hour at the initial astaxanthin accumulation stage, the mortality rate, immobility rate and average diameter of the algae cells were determined; during collection, the astaxanthin content and dry weight of the algae cells were determined, with results shown in Table 4 and Table 5.

TABLE 4
Mortality rate, immobility rate and average diameter of
algae cells under different illumination intensities

| | Total light | Mortality | Immobility | |
|---|---|---|---|---|
| Light | intensity | rate of | rate of | Average |
| intensity/density | (Lux) | cells (%) | cell (%) | diameter |
| 250 | 7500 | 1.5 | 90 | 27 |
| 300 | 9000 | 1.1 | 96 | 32 |
| 350 | 10500 | 1.1 | 96 | 32 |
| 400 | 12000 | 0.6 | 97 | 35 |
| 450 | 13500 | 5 | 98 | 34 |

TABLE 5
Astaxanthin content and dry weight of algal cells
under different illumination intensities

| | Astaxanthin | Dry weight |
|---|---|---|
| Light intensity/density | content (%) | (g/L) |
| 250 | 1.53 | 0.60 |
| 300 | 3.88 | 0.92 |
| 350 | 4.37 | 0.98 |
| 400 | 4.89 | 1.03 |
| 450 | 1.60 | 0.62 |

The results show that the light intensity suitable for the pre-stress treatment method provided by the present invention is that the light intensity of the solution of algae with the cell density of 1×10⁴ cells/mL is 300-400 Lux; with the light intensity being within the above light intensity, the cells have the low mortality rate, high immobility rate, large average diameter, and high productivity of astaxanthin; with the light intensity being lower than the above light intensity, the cells have the low immobility rate and the small average diameter; with the light intensity being higher than the above light intensity, the mortality rate is significantly increased. These are unfavorable to the production of astaxanthin, resulting in a serious decline in yield.

### Embodiment 6: Screening of Photoperoids

Photoperiod also affects the effect of prestress treatment: long illumination time causes an increase in mortality rate, which is not conducive to the production of the cells; and short illumination time causes poor photosynthesis and small cell diameter, which is also not conducive to the production of the cells. Therefore, a photoperiod suitable for the pre-stress treatment method provided by the present invention should be identified.

The embodiment shares a method with Embodiment 5, and their difference lies that a fixed light intensity is 12,000 Lux; the photoperiod L:D was adjusted to be 15:7, 16:8, 17:9, 18:6, and 19:5, respectively, so that the algal cells were subjected to prestress treatment for 36 hours and then entered into the astaxanthin accumulation stage in the same method as in Embodiment 1. At the 24th hour at the initial astaxanthin accumulation stage, the mortality rate, immobility and average diameter of the algae cells were determined; during collection, the astaxanthin content and dry weight of the algae cells were determined, with results shown in Table 6 and Table 7.

TABLE 6
Mortality rate, immobility rate and average diameter
of algae cells under different photoperoids

| | Mortality rate | Immobility rate | Average |
|---|---|---|---|
| Photoperoid | of cells (%) | of cell (%) | diameter |
| 15:7 | 1.1 | 91 | 26 |
| 16:8 | 0.6 | 97 | 35 |
| 17:9 | 1.4 | 97 | 36 |
| 18:6 | 2.7 | 98 | 36 |
| 19:5 | 5 | 98 | 32 |

TABLE 7
Astaxanthin content and dry weight of algal
cells under different photoperoids

| | Astaxanthin | Dry weight |
|---|---|---|
| Photoperoid | content (%) | (g/L) |
| 15:7 | 1.69 | 0.57 |
| 16:8 | 4.89 | 1.03 |
| 17:9 | 4.61 | 0.95 |
| 18:6 | 4.23 | 0.92 |
| 19:5 | 1.57 | 0.53 |

The results show that at the photoperiod of 16:8, 17:9 or 18:6, the cells have the low mortality rate, high immobility rate, large average diameter, high astaxanthin content, high dry weight, and good production effect of astaxanthin; therefore, the photoperiod is suitable for the pre-stress treatment method provided by the present invention; at the photoperiod of 16:8, the astaxanthin has the optimal production effect.

### Embodiment 7: Temperature and pH Control

The pre-stress treatment method provided by the present invention is designed to acclimatize the Haematococcus pluvialis from the logarithmic growth phase to the astaxanthin production cycle; therefore, the culture conditions need to be appropriately adjusted; the culture conditions being slightly adjusted will lead to the low productivity of the Haematococcus pluvialis in the astaxanthin production stage, and the culture conditions being excessively adjusted will lead to the soaring mortality rate of the Haematococcus pluvialis, with the possibility of yield reduction or even production failure. The temperature and pH control are discussed in the embodiment to optimize the pre-stress treatment method.

We use the same method as in Embodiment 5, and a difference between Embodiment 7 and Embodiment 5 lies in that a fixed light intensity is 12,000 Lux; the temperature is adjusted to be 24° C., 25° C., 26° C., 27° C., 28° C., 29° C., and 30° C. respectively, so that the Haematococcus pluvialis is subjected to prestress treatment for 36 hours and then enter into the astaxanthin accumulation stage in the same method as in Embodiment 1. At the 24th hour at the initial astaxanthin accumulation stage, the mortality rate, immobility and average diameter of the algae cells are determined; during collection, the astaxanthin content and dry weight of the algae cells are determined, with results shown in Table 8 and Table 9.

TABLE 8
Mortality rate, immobility rate and average diameter
of algae cells under different temperature

| Temperature | Mortality rate | Immobility rate | Average |
|---|---|---|---|
| (° C.) | of cells (%) | of cell (%) | diameter |
| 24 | 5.3 | 88 | 24 |
| 25 | 4.2 | 91 | 26 |
| 26 | 1.9 | 96 | 32 |
| 27 | 1.1 | 96 | 34 |
| 28 | 0.6 | 97 | 35 |
| 29 | 3.7 | 98 | 32 |
| 30 | 5.9 | 98 | 32 |

TABLE 9
Astaxanthin content and dry weight of algal
cells under different temperatures

| Temperature | Astaxanthin | Dry weight |
|---|---|---|
| (° C.) | content (%) | (g/L) |
| 24 | 1.54 | 0.55 |
| 25 | 2.07 | 0.60 |
| 26 | 4.29 | 0.97 |
| 27 | 4.70 | 1.00 |
| 28 | 4.89 | 1.03 |
| 29 | 2.21 | 0.63 |
| 30 | 1.50 | 0.52 |

The results show that at the temperature of 26° C.-28° C., the cells have the low mortality rate, high immobility rate, large average diameter, high astaxanthin content and high dry weight; at the temperature of 28° C., the astaxanthin has the optimal production effect; the high temperature will lead to the increased mortality rate and the low temperature will lead to the small average diameter, both which lead to the insufficiency of the astaxanthin content and dry weight of the algal cells.

The impact of the pH value of the culture system on the prestress effect of the Haematococcus pluvialis is further studied by using the same method as described above, and their difference that the temperature is controlled at 28° C. and only the culture system pH is adjusted to be 7.5, 8.0, 8.5, and 9.0 respectively by adjusting the carbon dioxide concentration of the culture system, so that the Haematococcus pluvialis is subjected to prestress treatment for 36 hours and then enters into the astaxanthin accumulation stage in the same method as in Embodiment 1. At the 24th hour at the initial astaxanthin accumulation stage, the mortality rate, immobility and average diameter of the algae cells are determined; during collection, the astaxanthin content and dry weight of the algae cells are determined, with results shown in Table 10 and Table 11.

TABLE 10
Mortality rate, immobility rate and average diameter
of algae cells under different pH values

| | Mortality rate | Immobility rate | Average |
|---|---|---|---|
| pH | of cells (%) | of cell (%) | diameter |
| 7.5 | 6.1 | 88 | 27 |
| 8.0 | 2.4 | 96 | 34 |
| 8.2 | 0.6 | 97 | 35 |
| 8.5 | 2.7 | 96 | 32 |
| 9.0 | 7.9 | 84 | 27 |

TABLE 11
Astaxanthin content and dry weight of
algal cells under different pH values

| | Astaxanthin | Dry weight |
|---|---|---|
| pH | content (%) | (g/L) |
| 7.5 | 2.41 | 0.64 |
| 8.0 | 4.58 | 0.98 |
| 8.2 | 4.89 | 1.03 |
| 8.5 | 4.53 | 0.97 |
| 9.0 | 2.26 | 0.60 |

The results show that at the pH of 8.0-8.5, the cells have the low mortality rate, high immobility rate, large average diameter, high astaxanthin content and high dry weight.

In addition, through a further test of pH within this interval, we find that pH=8.2 is most preferred, in this case, the cells have the mortality rate of only 0.6% and the optimal production effect of astaxanthin.

### Embodiment 8: Prestress Treatment Duration

The prestress treatment duration has a certain impact on the effect of prestress treatment: the short treatment time indicates the poor prestress effect, resulting in the increased mortality rate in the astaxanthin production stage; the long treatment time is easy to cause the poisonous effect on the algal cells, which directly leads to the death of the algal cells. The prestress treatment duration is studied in the embodiment.

The embodiment also shares the method with Embodiment 5, and their difference lies in that the fixed light intensity is 12,000 Lux, the prestress treatment duration is adjusted, and the mortality rate of the algal cells is detected, with results shown in Table 7.

TABLE 12
Effect of different prestress treatment durations
on the mortality rate of the algal cells

| | Mortality rate of |
|---|---|
| Duration (h) | algal cell (%) |
| 20 | 3.7 |
| 24 | 1.8 |
| 28 | 1.4 |
| 32 | 1.1 |
| 36 | 0.6 |
| 40 | 4.0 |

The results show that the suitable prestress treatment duration is 24-36 hours, and preferably 36 hours.

To sum up, the pre-stress treatment method for reducing a mortality rate of Haematococcus pluvialis provided by the present invention has the preferred implementation conditions: the characteristic peaks with the wavelength ranges of 430-490 nm and 620-700 nm as the spectra parameters, the characteristic peaks having the light intensity ratio of 0.7:1, the algal liquid with the cell density of 1×10⁴ cells/mL having a light intensity of 300-400 Lux, the photoperoid L:D of 16:8, the temperature controlled at 28° C., the pH controlled at 8.2, and the prestress treatment time of 36 hours; under the above conditions, the cells have the mortality rate reduced to 0.6%, and have the immobility rate of 97% and the average diameter of 35 m, which can effectively solve the problem of damage to cells in an existing culture method that the cells directly enter into the formal reddening promotion stage; the prestress treatment can reduce the mortality rate of the cells in an initial induction stage and ensure the productivity and process stability; after the above prestress treatment, the astaxanthin content of the algal cells is 4.89% during collection and much higher than that in the conventional method, and the dry weight of algal cells reaches 1.03 g/L, thereby significantly increasing the yield of astaxanthin; besides, this also avoids contamination from exogenous sources such as protozoa and microorganisms due to lysis of dead cells, greatly reducing the risk of production failure and improving product quality.

All the patents and publications mentioned in the description of the present invention indicate that these are public technologies in the art and can be used by the present invention. All the patents and publications cited herein are listed in the references, just as each publication is specifically referenced separately. The present invention described herein can be realized in the absence of any one element or multiple elements, one restriction or multiple restrictions, where such restriction is not specifically described here. For example, the terms “comprising”, “essentially consisting of” and “consisting of” in each example herein may be replaced by the rest two terms. The terms and expressions which have been used herein are descriptive rather than restrictive, and there is no intention to suggest that these terms and expressions in this description exclude any equivalents, but it is to be understood that any appropriate changes or modifications can be made within the scope of the present invention and appended claims. It can be understood that the embodiments described in the present invention are some preferred embodiments and features. Any person of ordinary skill in the art can make some modifications and changes according to the spirit of the description of the present invention. These modifications and changes are also considered to fall within the scope of the present invention and the scope limited by independent claims and dependent claims.

1. A pre-stress treatment method for reducing a mortality rate of Haematococcus pluvialis is provided, and the method includes: prestressing the Haematococcus pluvialis subjected to a logarithmic-growth proliferation stage, where external conditions for the pre-stress treatment method include characteristic peaks with wavelength ranges of 430-490 nm and 620-700 nm as spectra parameters.

2. The pre-stress treatment method according to claim 1, wherein a cell density of the Haematococcus pluvialis subjected to the logarithmic-growth proliferation stage is 3-4×10⁵ cells/mL.

3. The pre-stress treatment method according to claim 1, wherein the characteristic peaks with the wavelength ranges of 430-490 nm and 620-700 nm have a light intensity ratio of 0.1-1:1.

4. The pre-stress treatment method according to claim 1, wherein the external conditions for the pre-stress treatment method further comprise: an algal liquid with a cell density of 1×10⁴ cells/mL having a light intensity of 300-400 Lux.

5. The pre-stress treatment method according to claim 3, wherein the external conditions for the pre-stress treatment method further comprise: an algal liquid with the cell density of 1×10⁴ cells/mL having a light intensity of 300-400 Lux.

6. The pre-stress treatment method according to claim 1, wherein the external conditions for the pre-stress treatment method further comprise: a photoperoid L:D of 16-18:6-8.

7. The pre-stress treatment method according to claim 3, wherein the external conditions for the pre-stress treatment method further comprise: a photoperoid L:D of 16-18:6-8.

8. The pre-stress treatment method according to claim 1, wherein a photobioreactor is used as a light source for the pre-stress treatment method.

9. The pre-stress treatment method according to claim 3, wherein a photobioreactor is used as a light source for the pre-stress treatment method.

10. The pre-stress treatment method according to claim 1, wherein the external conditions for the pre-stress treatment method further comprise: a temperature of 26° C.-28° C. and a pH of 8.0-8.5.

11. The pre-stress treatment method according to claim 3, wherein the external conditions for the pre-stress treatment method further comprise: a temperature of 26° C.-28° C. and a pH of 8.0-8.5.

12. The pre-stress treatment method according to claim 1, wherein a treatment time for the pre-stress treatment method is 24-36 hours.

13. The pre-stress treatment method according to claim 3, wherein a treatment time for the pre-stress treatment method is 24-36 hours.

14. The pre-stress treatment method according to claim 1, wherein after the Haematococcus pluvialis is subjected to prestress treatment, Haematococcus pluvialis cells are capable to exhibit at least two of the following characteristics: original motile cells losing flagella and becoming immobile cells, thickening of walls of the cells, and accumulation of a small amount of astaxanthin in the centers of the cells.

15. The pre-stress treatment method according to claim 3, wherein after the Haematococcus pluvialis is subjected to prestress treatment, Haematococcus pluvialis cells are capable to exhibit at least two of the following characteristics: original motile cells losing flagella and becoming immobile cells, thickening of walls of the cells, and accumulation of a small amount of astaxanthin in the centers of the cells.

16. The pre-stress treatment method according to claim 9, wherein after the Haematococcus pluvialis is subjected to prestress treatment, Haematococcus pluvialis cells are further capable to exhibit at least one of the following characteristics: the cells changing from an original water drop shape to a spherical shape, and the increase of the cells in volume and diameter.

17. The pre-stress treatment method according to claim 1, wherein the light intensity is 12,000 Lux, a photoperoid L:D is 16:8, a temperature is controlled at 26° C.-28° C., and a pH is controlled at 8.0-8.2; in a specific embodiment, the temperature is 28° C. and the pH is controlled at 8.2.
